(19) United States
(12) Patent Application Publication
(10) Pub. No.: US 2025/0362363 A1
(43) Pub. Date: Nov. 27, 2025

(54) SYSTEMS AND METHODS FOR PLANAR CORTICAL MAGNIFICATION MEASUREMENT

(71) Applicants: ARIZONA BOARD OF REGENTS ON BEHALF OF ARIZONA STATE UNIVERSITY, Scottsdale, AZ (US); NEW YORK UNIVERSITY, New York, NY (US)

(72) Inventors: Yujian XIONG, Scottsdale, AZ (US); Zhong-Lin LU, New York, NY (US); Yalin WANG, Scottsdale, AZ (US)

(21) Appl. No.: 19/289,871

(22) Filed: Aug. 4, 2025

Related U.S. Application Data
(63) Continuation of application No. PCT/US24/14617, filed on Feb. 6, 2024.
(60) Provisional application No. 63/483,382, filed on Feb. 6, 2023.

(51) Int. Cl. G01R 33/56; A61B 5/00; A61B 5/055; G01R 33/48; G06T 3/06; G06T 7/00; G16H 30/20; G16H 30/40

(52) U.S. Cl. CPC G01R 33/5608; A61B 5/055; A61B 5/4064; G01R 33/4806; G06T 3/06; G06T 7/0012; G16H 30/20; G16H 30/40; G06T 2207/30016

(57) ABSTRACT

Exemplary systems and methods utilize topological smoothing and optimal transportation to estimate a cortical magnification factor. Via these methods, medical practitioners can obtain improved information regarding visual system of a patient.

### CROSS-REFERENCE TO RELATED APPLICATION

This application is a continuation of PCT Application No. PCT/US2024/014617 filed Feb. 6, 2024, now WIPO Publication No. WO/2024/167920 entitled “SYSTEMS AND METHODS FOR PLANAR CORTICAL MAGNIFICATION MEASURMENT.” PCT Application No. PCT/US2024/014617 claims priority to, and the benefit of, U.S. Provisional Patent Application Ser. No. 63/483,382 filed Feb. 6, 2023 entitled “Systems and Methods for Planar Cortical Magnification Measurement.” The foregoing applications are hereby incorporated by reference in its entirety for all purposes, including but not limited to those portions that specifically appear hereinafter, but except for any subject matter disclaimers or disavowals, and except to the extent that the incorporated material is inconsistent with the express disclosure herein, in which case the language in this disclosure shall control.

### FEDERALLY SPONSORED RESEARCH OR DEVELOPMENT

This invention was made with government support under R01 EY032125 awarded by the National Institutes of Health. The government has certain rights in the invention.

### TECHNICAL FIELD

The present disclosure relates to systems and methods to measure and illustrate cortical magnification factors of the visual cortex of the human brain.

### BACKGROUND

The human visual system devotes varying amounts of neural resources in the visual field. The cortical magnification factor (CMF) is a popular measure of the ratio of the cortical area devoted to each region of the visual field. In many prior approaches, Blood Oxygenation Level Dependent (BOLD) fMRI activation data was used to generate retinotopic maps, which can be used to estimate CMF. However, due to the low signal-to-noise ratio and spatial resolution, CMF is currently estimated only as a function of retinal eccentricity. Accordingly, improved approaches are desirable.

### SUMMARY

In various embodiments, a computer-based method for planar cortical magnification measurement comprises: obtaining a first input comprising a visual stimuli and a coordinate system of a visual field; obtaining a second input comprising a structural magnetic resonance imaging (MRI) image and functional MRI (fMRI) scans; storing the first input and the second input in an electronic database; pre-processing, by a processor, the fMRI scans; calculating, by the processor, a reconstructed cortical surface with projected fMRI activation and surface extraction; calculating, by the processor, retinotopic maps comprising receptive center and population receptive field (pRF) size of each vertex, together with projection of the cortex onto a 2D planar disk with optimal transportation that preserves local area; and applying, by the processor, topological smoothing to the calculated retinotopic maps to obtain smoothed retinotopic maps.

The method may comprise applying, by the processor, a 1-ring patch method to the smoothed retinotopic maps. The method may comprise providing, to a medical provider and over an electronic communications network, the planar cortical magnification measurement. The method may comprise utilizing, by the medical provider, the planar cortical magnification measurement to facilitate a medical diagnosis. The calculating the reconstructed cortical surface may comprise acquiring a 3D cortical surface mesh. The calculating retinotopic maps may comprise utilizing an experiment stimulus and fMRI signal into a pRF analysis module operative on the processor to obtain initial pRF results. The applying topological smoothing may utilizes the initial pRF results as inputs. The reconstructed cortical surface may comprise a left hemisphere and a right hemisphere. The calculating retinotopic maps and the applying topological smoothing may be performed separately for the left hemisphere and the right hemisphere. The method may be implemented as software code operative on the processor.

The foregoing features and elements may be combined in various combinations without exclusivity, unless expressly indicated herein otherwise. These features and elements as well as the operation of the disclosed embodiments will become more apparent in light of the following description and accompanying drawings.

### BRIEF DESCRIPTION OF THE DRAWINGS

With reference to the following description and accompanying drawings:

FIGS. 1A and 1B illustrate a processing pipeline for planar cortical magnification measurement in accordance with various exemplary embodiments;

FIG. 2 illustrates evaluation of cortical magnification factor in accordance with various exemplary embodiments;

FIG. 3A illustrates an exemplary planar CMF result of a cluster (left-above), in accordance

with various exemplary embodiments;

FIG. 3B illustrates an exemplary planar CMF result of a cluster (right-below), in accordance with various exemplary embodiments;

FIG. 3C illustrates an exemplary planar CMF result of a cluster (dual-centralized), in accordance with various exemplary embodiments;

FIG. 3D illustrates an exemplary planar CMF result of a cluster (centralized), in accordance with various exemplary embodiments;

FIG. 3E illustrates an exemplary planar CMF result of a cluster (below-centralized), in accordance with various exemplary embodiments;

FIG. 4A illustrates correlation between CMF and pRF size across eccentricity p, in accordance with various exemplary embodiments;

FIG. 4B illustrates correlation between CMF and pRF size across polar angle Θ, in accordance with various exemplary embodiments; and

FIGS. 5A through 5E illustrate comparisons between planar CMF and inverse of pRF size 1/σ for various subjects, in accordance with various exemplary embodiments.

### DETAILED DESCRIPTION

The following description is of various exemplary embodiments only, and is not intended to limit the scope, applicability or configuration of the present disclosure in any way. Rather, the following description is intended to provide a convenient illustration for implementing various embodiments including the best mode. As will become apparent, various changes may be made in the function and arrangement of the elements described in these embodiments without departing from the scope of the present disclosure.

In various exemplary embodiments, principles and techniques disclosed herein may be combined with, integrated with, operable on, or otherwise utilized in connection with various computing and/or imaging systems. For example, magnetic resonance imaging (MRI) machines such as fMRI machines offered by Philips, GE, and Siemens may be utilized to obtain suitable input data. In various exemplary embodiments, data from a Siemens fMRI machine may be utilized. In various exemplary embodiments, exemplary pipelines may be constructed utilizing Matlab software offered by Mathworks Corporation, operative on a PC workstation. It will be appreciated that principles of the present disclosure improve the functioning of a computing device by enabling better spatial resolution of resulting images, eliminating topology violations in retinopic mappings and thus increasing the accuracy of CMF measurement, and so on. Moreover, the smoothing of retinotopic mapping will help to accelerate and reduce computation for most measurements on brain cortex, because it provides topological correct results which could distinguish brain boundaries and regions, allowing users to choose desired portion of brain for more accurate and faster measurements. Additionally, the smoothed retinotopic pRF results may provide better medical analysis of human cortex compared to ordinary pRF results, because the latter one has topological violations which will interfere retinotopy diagnosis on brain cortex, while exemplary embodiments provide more accurate results. Yet further, exemplary embodiments can also improve functioning of computing devices by helping reduce data and computation resources in deep learning models which process fMRI data, for example, many popular models aims to reconstruct visual inputs from fMRI. Exemplary embodiments can distinguish brain cortex regions with topological correct results and tell the relation between vision and cortex region, allowing those models to choose appropriate data instead of using the entire data set and thus reducing the computational burden and consequent expenditure of energy on unnecessary data processing.

In clinical settings, doctors may require an illustration of CMF in the visual cortex. CMF is currently estimated only as a function of retinal eccentricity. An exemplary pipeline provides smoothing tools, topology correction tools, measurement tools, and illustration tools is needed in this mission. By comparing the results between healthy and unhealthy individuals, doctors can detect or distinguish diseases and make a diagnosis and/or prognosis.

In various exemplary embodiments, disclosed is a novel algorithm that can precisely measure and illustrate cortical magnification factors in the visual cortex. The measurements can be used to quantify CMF in the human visual field and show their individual differences, normal subjects and people with visual disease. Moreover, the principles of the present disclosure build a solid CMF measurement after years of exploring computational conformal geometry and brain MRI recordings. By combining geometry and neuroscience, exemplary approaches disclosed herein successfully removed the topology violations, improved the resolution, and finally achieved a thorough description of human CMF which was not possible before. These concepts provide a novel goal and tool for companies and researchers who want to dig deep into the human visual system.

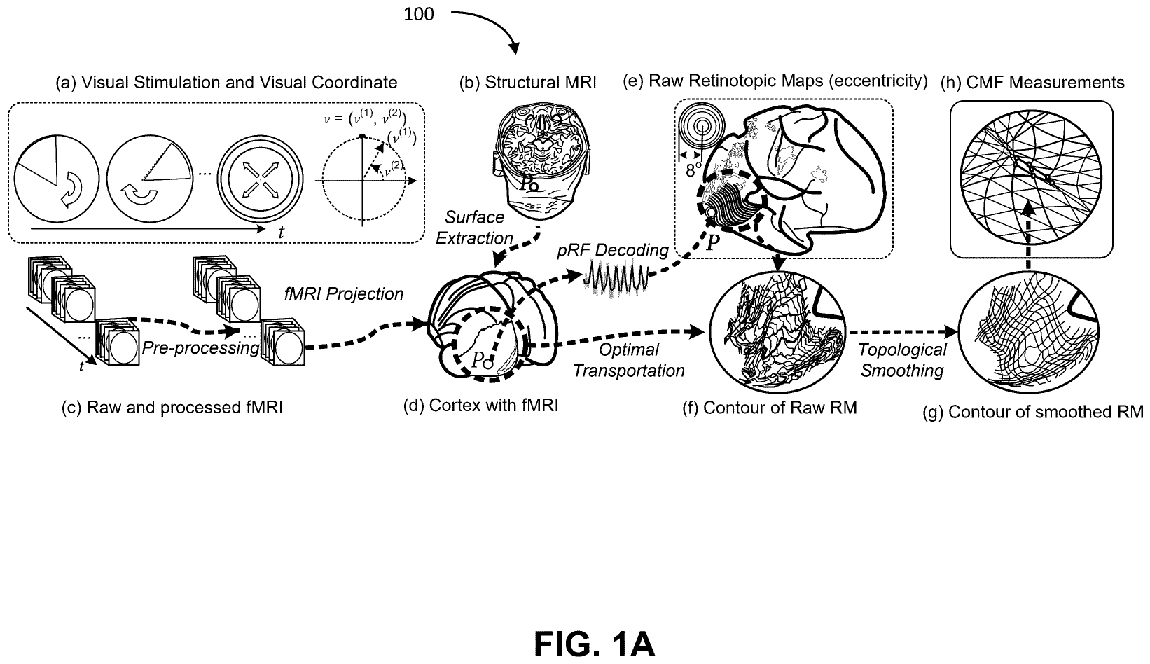

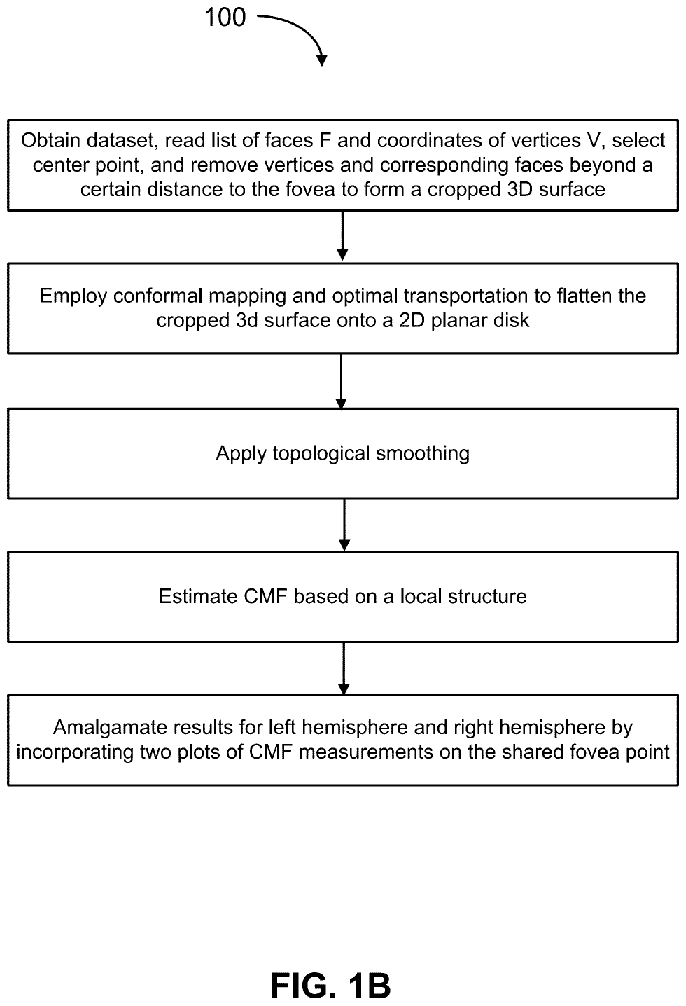

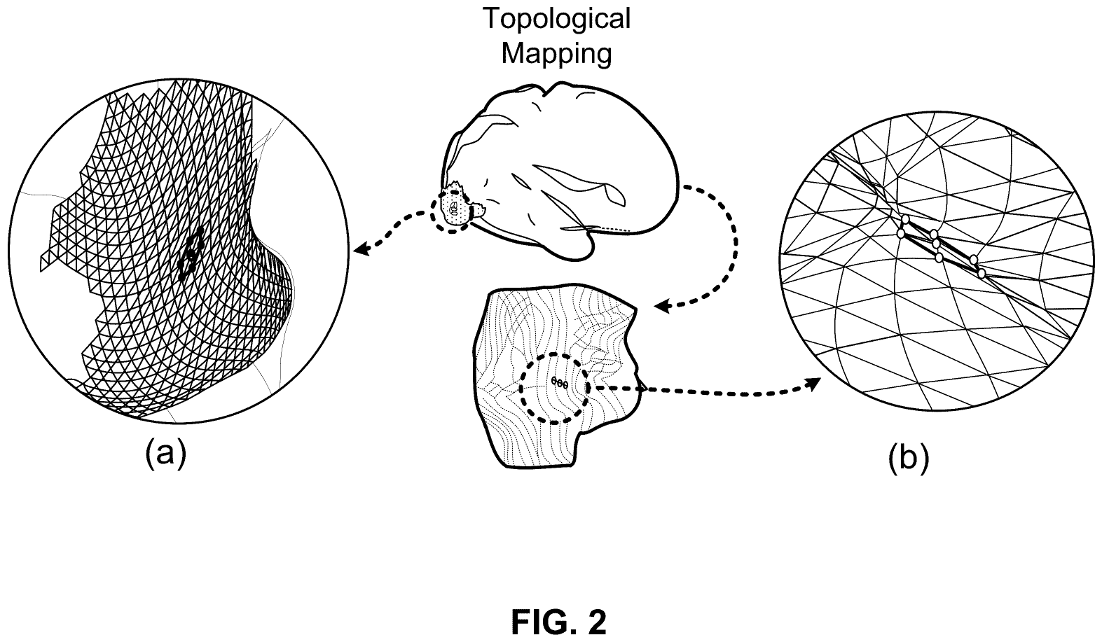

With reference now to FIGS. 1A, 1B, and 2, in various exemplary embodiments, an exemplary method pipeline starts with fMRI pre-processing, population receptive field (pRF) decoding. After that, an exemplary method comprises recalculating the parametric coordinate of each voxel by optimal mass transportation on the cortical surface that preserves local area, in order to modify the raw retinotopic maps (RM) from pRF decoding. Thereafter, there still exist many topological violations. A contour is drawn on the raw RM, so topological smoothing is applied to fix such topological violations, which make it possible for an exemplary method to run 1-ring patch method for planar CMF measurement.

The CMF can be estimated based on a local structure, for example a vertex dual structure or the vertex 1-ring patch. The vertex 1-ring is a patch around the center vertex so that any vertex in the patch can reach the center vertex by “walking” within one edge. After smoothing, since topology is preserved, an exemplary method can calculate the enclosed visual area. Eventually, the CMF for the center point may be estimated by dividing the cortical surface area by the visual field area within the vertex 1-ring patch.

In a further step, an exemplary method contemplates merging the CMF results from both the left and right hemispheres, to obtain a combined perceptual concentration on the full visual field. In a validation exercise, the complete pipeline of an exemplary method was tested on the Human Connectome Project 7T fMRI dataset and produced high-quality results.

Specifically, as compared to prior approaches, exemplary methods disclosed herein have the following advantages compared to current alternatives: (1) zero topology violations in retinotopic mappings which allow more accurate CMF measurement; (2) better spatial resolution in retinotopic mapping and CMF results; (3) achieved thorough CMF results in 2D planar visual field, instead of ordinary function of only retinal eccentricity; and (4) excellent illustration of CMF showing human CMF and their differences.

With reference now to FIGS. 1 through 5E and in accordance with various exemplary embodiments, algorithms and systems for evaluating CMF are disclosed. As is known, the mammalian visual cortex contains multiple representations of different visual field areas. It has been an attracting topic to obtain retinotopic representation through activity and anatomy of the visual cortex. In many studies, Blood Oxygenation Level Development (BOLD) fMRI activation data was used to provide great analysis on retinotopic mapping, along with the population receptive field (pRF) model (See FIG. 1A), which estimates the center and size of the receptive field that is monitored by certain voxel on the cortical surface. Retinotopic maps generated by such methods dramatically improved our knowledge of the human visual system. However, because the pRF model was designed to fit an fMRI signal on the cortex, it is difficult to link the retinotopic maps with the actual visual behaviours of subjects. Accordingly, exemplary approaches herein provide for new measurements to illustrate visual behavior through retinotopic maps.

The human primary visual cortex is an ideal system to investigate the relationship of cortex anatomy and visual perception. Retinotopic mapping gives the projection of a 3D brain cortex onto its responsible area on visual field. Intuitively, a larger responsible area means less visual acuity for a fixed area of cortex. Accordingly, prior researchers have introduced cortical magnification factor (CMF), the ratio between the distances of two points on the cortical surface and the corresponding points in the visual field, as one of the measurements of this phenomenon. CMF not only shows the relationship between visual acuity and cortical structure, it also changes asymmetrically as a function of eccentricity and polar angle, and possess large differences among individuals. Accordingly, accurate and area-specific measurements of CMF are highly desirable.

Prior template-based pRF methods are able to calculate the retinotopic maps to acquire visual cortex area and corresponding receptive center and receptive size from fMRI signals on a voxel-by-voxel basis. However. due to low signal-to-noise (SNR) ratio and low spatial resolution, typical pRF results are usually not topological, especially in those areas that are close to the fovea. It will be appreciated that CMF and other visual-cortical measurements require the retinotopic maps to preserve local neighborhood geometric relationships, i.e., neighboring points on the cortical surface should have neighboring retinal visual coordinates; referred to herein as topology preserving.

Due to these and other difficulties with such pRF methods with topological violations, people often measure CMF approximately, for example as a function of single parameter (eccentricity or polar angle). Moreover, prior approaches are only able to show asymmetry by comparing summed large-scope CMF in different directions, thus lacking the illustration of detailed area-specific CMF behavior in the visual field, particularly near the fovea. However, in order to obtain the desired detailed measurements of CMF, it is necessary to fix topological violations in the pRF results. Because topological violations break the requirement of the visual system's hierarchical organization, in these circumstances each visual area may not represent a certain portion of retina.

During the past few decades, many researchers have made significant efforts on improving the quality of fMRI recording, enhancing the accuracy and stability of pRF solution, and correcting or reducing the topological violations. But the topology preserving was not fully guaranteed until work by the inventors herein, which fully solved the topological violations in a topological-smoothing algorithm. With the topological violations corrected, it makes it possible for exemplary approaches to do accurate visual-related quantification of retinotopic maps, such as CMF measurements. In order to measure CMF directly on the 2D planar visual field, disclosed herein is an exemplary approach referred to as the 1-ring patch method.

Exemplary processing pipelines disclosed herein improve the accuracy and credibility when solving for the visual coordinates of receptive center. The improved pRF solution gives the exemplary approaches a high level of confidence and exactness regarding the final CMF measurements.

It is now understood that there exists an approximately negative correlation between pRF size and CMF in particular area on the visual field. That is, locations that have better visual acuity are usually accompanied with larger CMF and smaller pRF size. This phenomenon is intuitive, but prior reports were still in large-scale, and could not sufficiently identify the correlation in specific area(s) on the 2D planar visual field. By use of an exemplary pipeline as set forth in various exemplary embodiments, users can overlap planar CMF results onto the pRF solution, revealing the correlation which was not widely inspected before.

Exemplary algorithms make it possible to measure CMF through BOLD based fMRI exactly on the 2D planar visual field, instead of approximation on some certain direction. Moreover, the exemplary systems and methods are able to make illustration of CMF for each individual, which helps providers to characterize people's visual concentration behavior and locate their individual difference. For example, some subjects may have better visual acuity in the lower region under the fovea, while others may observe more clearly in the upper region above the fovea.

For validation of exemplary embodiments, the pipeline was applied on real retinotopic data from the Human Connectome Project (HCP), and achieved results in line with expectations. Our CMF measurement pipeline helps to address the problems about human visual concentration behaviors, and sheds light on how to illustrate functional visual differences between healthy and unhealthy subjects.

In various exemplary embodiments, exemplary data processing and algorithm approaches were performed on Human Connectome Project (HCP) retinopic data, which is publicly available at (https://www.humanconnectome.org/study/hcp-young-adult). pRF results and stimuli and analysis packages utilized are publicly available at (https://osf.io/bw9ec/)2 or (https://balsa.wustl.edu/study/show/9Zkk). The utilized retinotopic dataset consists of retinotopy data from 181 subjects who participated in retinotopic mapping experiments with 7T fMRI scanning. The pRF results of the published dataset contains fitted pRF results of retinotopy and atlases of cortical surface for each individual.

### Purpose

The mapping between visual field and cortical space indicates how a person sees the world. When a visual field is represented by a large area of cortical space, that individual would have more concentration on this field, which could be interpreted as a larger acuity.

Typically, as eccentricity increases across visual field, neural receptive field (RF) and population receptive field (pRF) sizes increase, and cortical magnification factor (CMF) decreases, which imply a keen perception near the fovea and a coarser perception outside. There is also evidence regarding differences between perception performance across different polar angles. At certain eccentricity, exemplary approaches observe different perception performance and asymmetries between the left, right, upper, or lower vision area. Moreover, recent fMRI studies also suggest large differences in these perceptual behaviors between individuals.

Disclosed herein is a novel method (or “pipeline”) to clearly measure CMF on the whole visual field. The area-CMF is defined as the area of a small patch on the cortex divided by the area of the patch correspondingly in visual field (with unit of mm2/deg2), which is slightly different from the line-CMF (with unit of mm/deg) estimated based on the banded visual region.

There may be variations in both pRF size and CMF across both eccentricity and polar angle, which could be interpreted as certain concentration area in the planar visual field. Exemplary pipelines successfully measure the planar CMF, and shows different concentration area in each subjects' visual field.

### Method

Besides receptive field size and population receptive field (pRF) size, cortical magnification factor (CMF) is also a popular measurement on visual acuity and cortex concentration. However, prior methods calculate CMF by the ratio between the distance of two points on the cortical surface and the distance of corresponding points in the visual field. These linear measurements only exhibit trends of CMF under a narrow range of eccentricity but a wide range of polar angles. In order to achieve thorough measurement of CMF across the whole visual field, exemplary embodiments utilize a pipeline 100 to obtain topological preserved retinotopic maps, and calculate planar CMF by dividing the area of certain patches on the cortical surface and corresponding area on the visual field with an exemplary 1-ring patch method.

In one example, pipeline 100 was applied to the V1, V2, and V3 area in the Human Connectome Project 7T dataset. The process utilized the data of 181 subjects from the HCP 7T MRI scans. In various exemplary embodiments, an exemplary method comprises fMRI pre-processing and projections onto cortex surface, then applying compressive pRF decoding to obtain raw retinotopic maps. Thereafter, the exemplary method utilizes optimal transportation to re-calculate the polar angle coordinates of each voxel on the flattened surface, and utilizes topological smoothing to fix topological violations in the retinotopic maps. The smoothed retinotopic maps are feasible for planar CMF measurement with 1-ring patch methods. The exemplary method further comprises merging the results of the left and right hemisphere for each subject, in order to demonstrate the properties of CMF and their individual difference.

### Pipeline Detail

An exemplary pipeline 100 is shown in FIGS. 1A and 1B. In the HCP dataset, the visual stimuli consist of rotating wedges, expanding or shrinking rings, and moving bars. The carriers of the stimuli (FIG. 1A, part a) are made of dynamic color textures that can better activate corresponding neurons on visual cortex. A point in the visual field is denoted by v=(v(1), v(2)) ∈ R2, where v(1) is the eccentricity (distance to the fovea in degrees of visual angle), and v(2) is the polar angle relative to the positive horizontal line (FIG. 1A). The corresponding Cartesian coordinate of a point is denoted as ρ=(ρX, ρY) ∈ R2. Both high-quality structural MRI and fMRI scans were acquired by the HCP group (FIG. 1A, parts b and c).

An exemplary pipeline 100 starts with fMRI pre-processing, compressive population receptive field decoding. Thereafter, pipeline 100 recalculates the parametric coordinate of each voxel by optimal mass transportation on the cortical surface, in order to modify the raw retinotopic maps (RM) from pRF decoding (FIG. 1A, portion (e)). At this point, there still exist many topological violations (FIG. 1A, portion (f)) if a contour is drawn on the raw RM, so topological smoothing is applied to fix such topological violations. With the violations addressed, it is now suitable for the system to run a 1-ring patch method for planar CMF measurement.

The Cortical Magnification Factor (CMF) can be estimated based on a local structure, for example a vertex dual structure or the vertex 1-ring patch. The vertex 1-ring is a patch around the center vertex so that any vertex in the patch can reach the center vertex by “walking” within one edge. In FIG. 2, portion (a), illustrated is the vertex (center red point) and its 1-ring patch (consisted of the enclosed blue polygon). After smoothing, each point on the cortical surface within the V1-V3 complex has a visual coordinate, i.e., the exemplary method has a topological mapping for V1, V2, and V3. In FIG. 2, portion (b), it can be seen that the 1-ring patch is also mapped as a 1-ring patch. Because topology is preserved, exemplary approaches can calculate the enclosed visual area. Eventually, the CMF for the center point is estimated by dividing the visual field area by cortical surface area within the vertex 1-ring patch. It will be appreciated that only a topological result can be used to estimate CMF in this way; otherwise, the visual area will be very noisy (a known problem of non-topological results).

In various exemplary embodiments, to generate an outcome of an exemplary topological pipeline, the system modeled the topological condition and generated topological and smooth retinotopic maps. The Beltrami coefficient, a metric of quasiconformal mapping, was used to define the topological condition. Exemplary embodiments utilize a mathematical model to quantify topological smoothing as a constrained optimization problem and elaborate an efficient numerical method to solve it. As a result, exemplary embodiments feature improved boundary delineation. Conventional methods have not fully considered topological constraints for multiple regions in retinotopic smoothing, which was not able for clear planar CMF measurements. In contrast, the novel algorithm herein made the retinotopic maps from BOLD fMRI topological and consistent with results from neurophysiology, which improved the quality of retinotopic mapping and built a solid foundation for the 1-ring patch measurements.

With continued reference to FIGS. 1A and 1B, in various exemplary embodiments a pipeline to obtain topological preserved retinotopic maps is presented. The pipeline may be applied to various datasets, for examine the Human Connectome Project (HCP) retinotopic data (available at humanconnectome.org/study/hcp-young-adult), which includes pRF coordinates, visual perception centers, size, and R2 values. Other suitable datasets may be utilized, for example other surface mesh and corresponding pRF results in one or more of the following formats: cortical surface mesh from Freesurfer (github.com/freesurfer/freesurfer), cortical surface mesh and pRF results from Ciftify toolbox (github.com/edickie/ciftify), or the like. Moreover, various additional software packages may be utilized in the pipeline, for example the following Matlab packages: Freesurfer Reader, Gifti (for reading data in gii format), fast marching toolbox (for geodesic distance computation), pRF-decoder (for pRF decoding and evaluation; see kendrickkay.net/analyzePRF), and Geometry Processing Package (tools for reading, writing, and processing mesh data, including boundary specification and harmonic map computation usable in conformal mapping of the cortical surface). These packages may be obtained from zenodo.org/records/6774948).

With continued reference to FIGS. 1A and 1B, in various exemplary embodiments a pipeline 100 is illustrated. In the HCP dataset, the visual stimuli consist of rotating wedges, expanding or shrinking rings, and moving bars. The carriers of the stimuli (FIG. 1A, part (a)) are made of dynamic color textures that can better activate corresponding neurons on the visual cortex. A point is denoted in the visual field by v=(v (1), v(2)) ∈ R2, where v(1) is the eccentricity (distance to the fovea in degrees of visual angle), and v(2) is the polar angle relative to the positive horizontal line.

Step 1: When utilizing prepared data from the previous session, users can commence directly with the surface cortex (FIG. 1A, portion (d)). Initially, pipeline 100 reads the list of faces F and coordinates of vertices V, selecting a center point that approximately corresponds to the fovea of the primary visual cortex V1. In the exemplary pipeline, designate v0=V[23452] as the fovea point on the HCP 53k cortical surface mesh. Subsequently, pipeline 100 removes all vertices and corresponding faces situated far from the fovea point by assessing their geodesic distance to the fovea point. Finally, pipeline 100 employs conformal mapping and optimal transportation to flatten the cropped 3D surface onto a 2D planar disk (FIG. 1A, portion (f)). This process provides the coordinates of each vertex on the 2D domain, facilitating the mapping of their pRF parameters onto the 2D planar disk.

Step 2: At this point, numerous topological violations may persist (See FIG. 1A, portion (f)) when contouring the raw pRF parameters. Therefore, in pipeline 100, topological smoothing is applied to rectify these violations. By inputting the original pRF parameters and vertex coordinates on the 2D planar disk (FIG. 1A, portions (e) and (f)), the exemplary topological smoothing algorithm generates corrected pRF parameters, effectively mitigating or even eliminating topology violations.

Step 3: In pipeline 100, the estimation of the CMF can be conducted based on a local structure, for example a vertex dual structure or the vertex 1-ring patch, owing to the correctness of surface contour topology (FIG. 1A, portion (g)). The vertex 1-ring refers to a patch surrounding the center vertex, allowing any vertex within the patch to connect to the center vertex through one edge “walk”. In FIG. 2, portion (a), the vertex (center red point) and its 1-ring patch (comprising the enclosed blue polygon) are illustrated. With the smoothed pRF parameters, each point on the cortical surface within the V1-V3 complex possesses a visual coordinate, establishing a topological mapping for V1, V2, and V3. FIG. 2, portion (b), demonstrates that the 1-ring patch is also mapped as a 1-ring patch, maintaining topology and enabling the calculation of the enclosed visual area. Eventually, the CMF for the center point is estimated by dividing the visual field area by the cortical surface area within the vertex 1-ring patch. It is important to note that only a topological correct pRF result can be employed for CMF estimation in this manner; otherwise, the visual area may be excessively noisy, presenting a challenge associated with non-topological results.

Step 4: In pipeline 100, the processing and measurement of the CMF are performed separately for the left hemisphere and the right hemisphere. In the concluding step, pipeline 100 amalgamates their results by incorporating two plots of CMF measurements on the shared fovea point. Subsequently, the merged results are applied to downstream tasks, such as clustering, to discern individual visual acuity preferences.

Table 1 illustrates performance of an exemplary method against various prior approaches.

TABLE 1
Comparison of CMF measurement methods

| Methods | # of outlier point | Smoothing MSE |
|---|---|---|
| 1. Linear CMF | 89 | 21.3 |
| 2. Area Preserving | 267 | 18.7 |
| 3. Disclosed | 124 | 13.0 |

In a final step, in various exemplary embodiments the CMF results from the left and right hemisphere are combined to obtain a comprehensive perceptual concentration on the whole visual field.

### Results

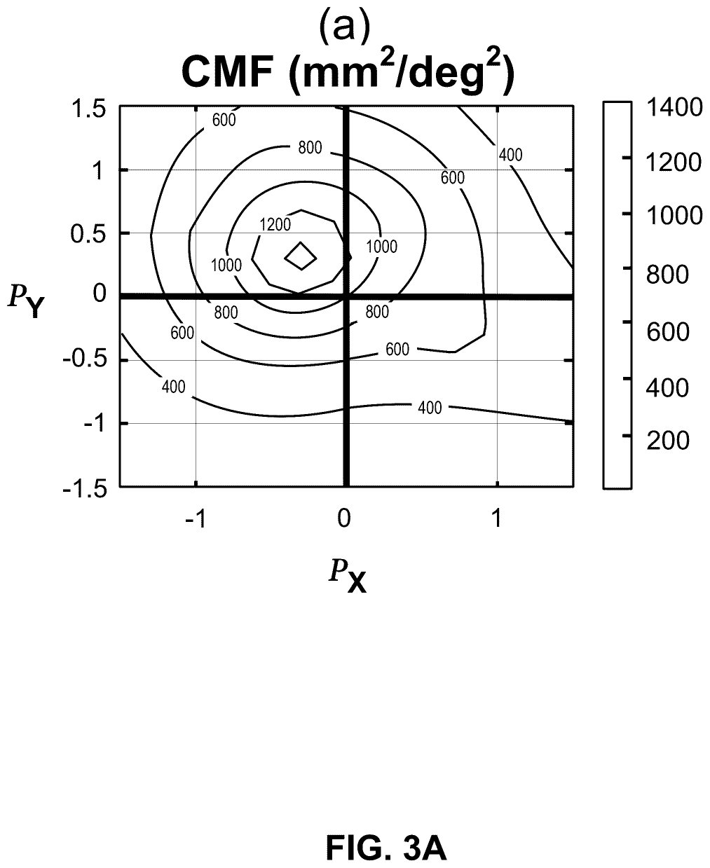

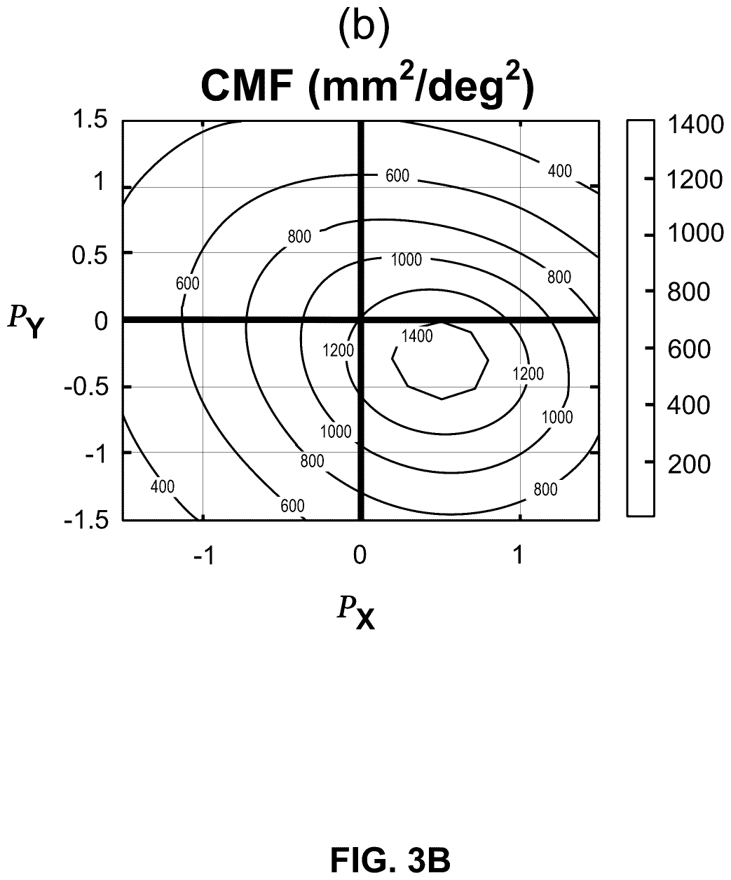

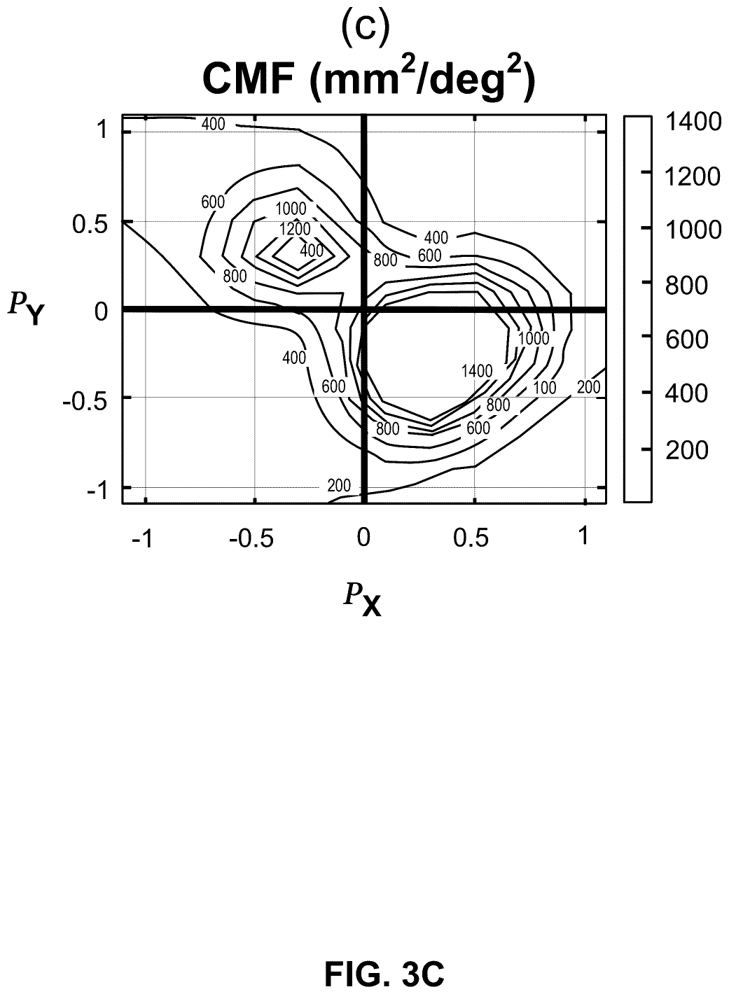

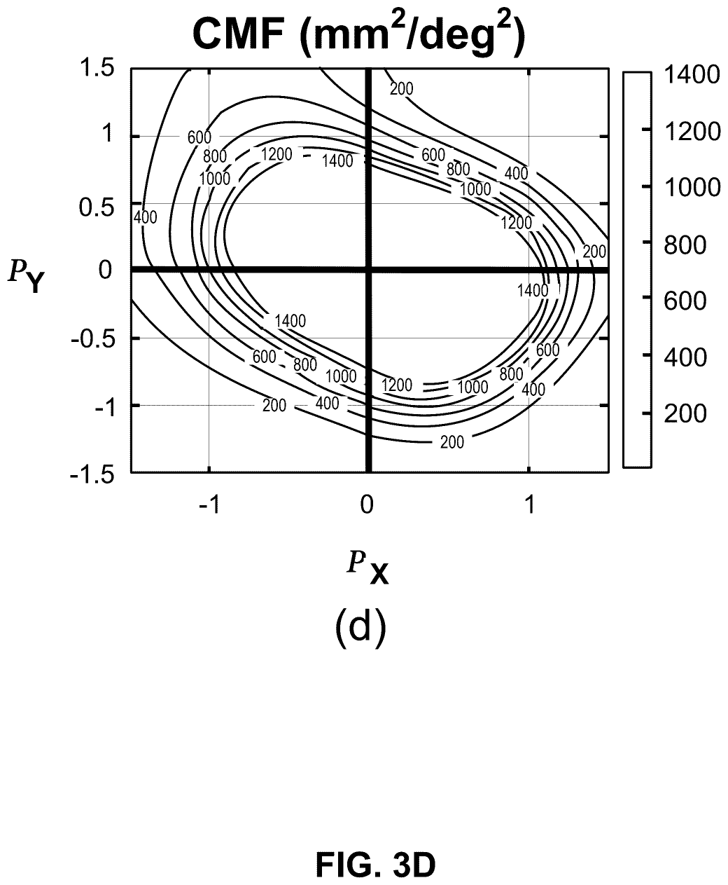

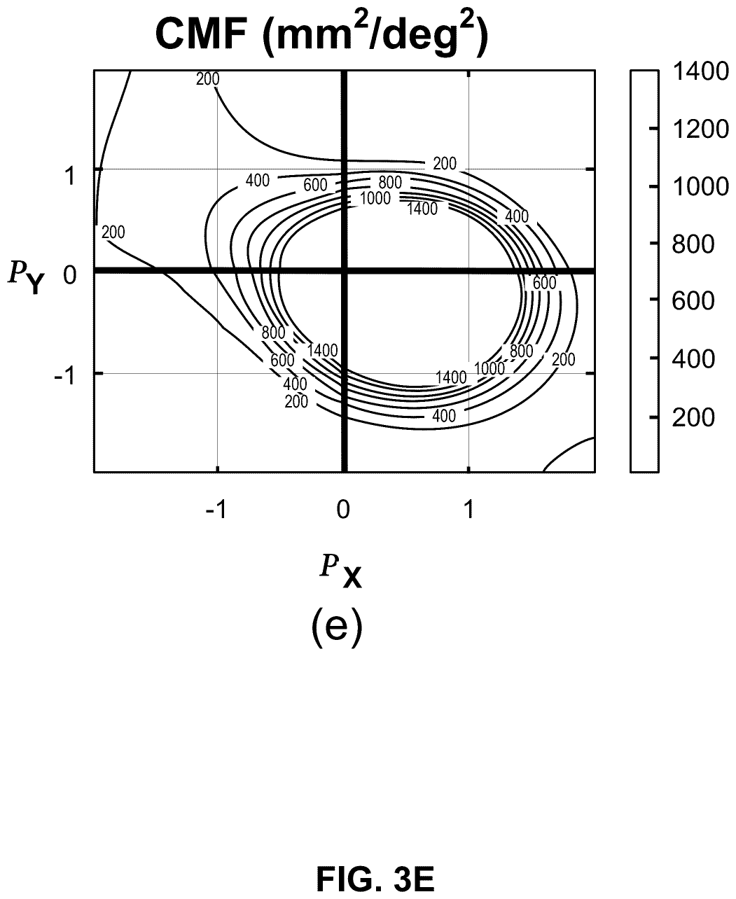

Use of an exemplary method uncovered that the CMF of most subjects centralized near the fovea area, while their asymmetries exist across polar angle and differ from each other. Exemplary methods apply K-means clustering on the CMF results, and obtained best elbow of K-value at 5. In these 5 clusters, people's concentration area lies separately, in one of left-above, right-below, centralized, below-centralized, or dual-centralized (some subjects show 2 concentration areas). See FIGS. 3A-3E.

Table 2 illustrates the number of subject in the example data falling into each cluster. It can be seen that people preserve focus but have different concentration behavior near fovea.

TABLE 2
Number of subjects for each cluster

| Clusters | # of subjects |
|---|---|
| a. left-above | 32 |
| b. right-below | 26 |
| c. dual-centralized | 11 |
| d. centralized | 47 |
| e. below-centralized | 68 |

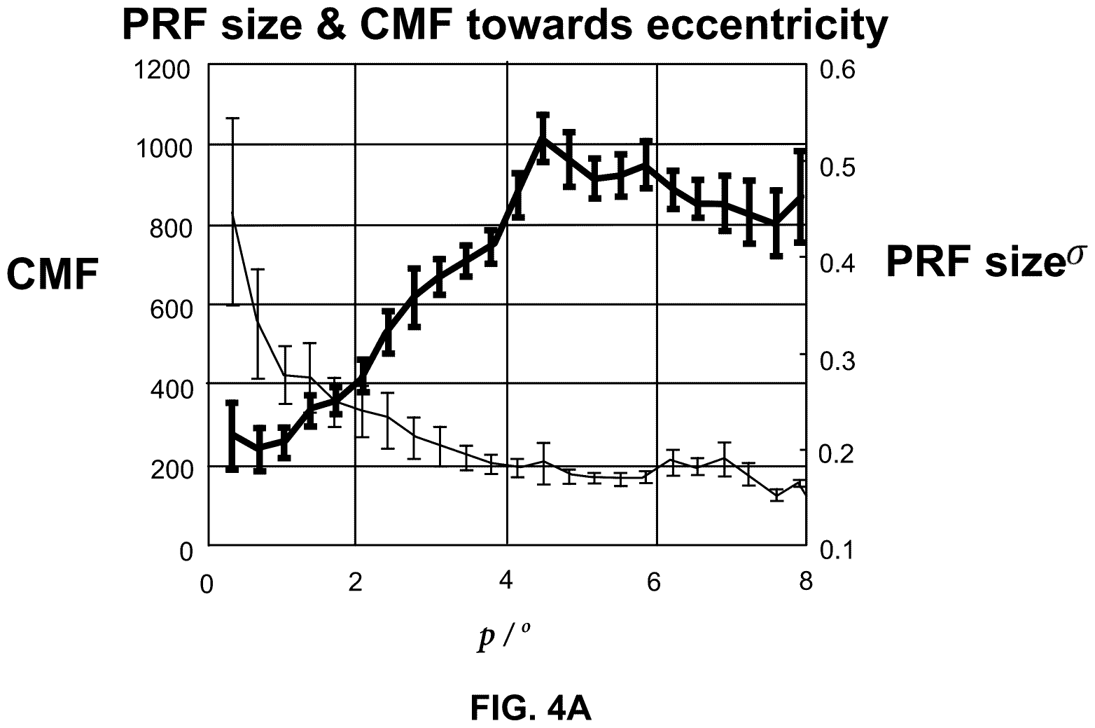

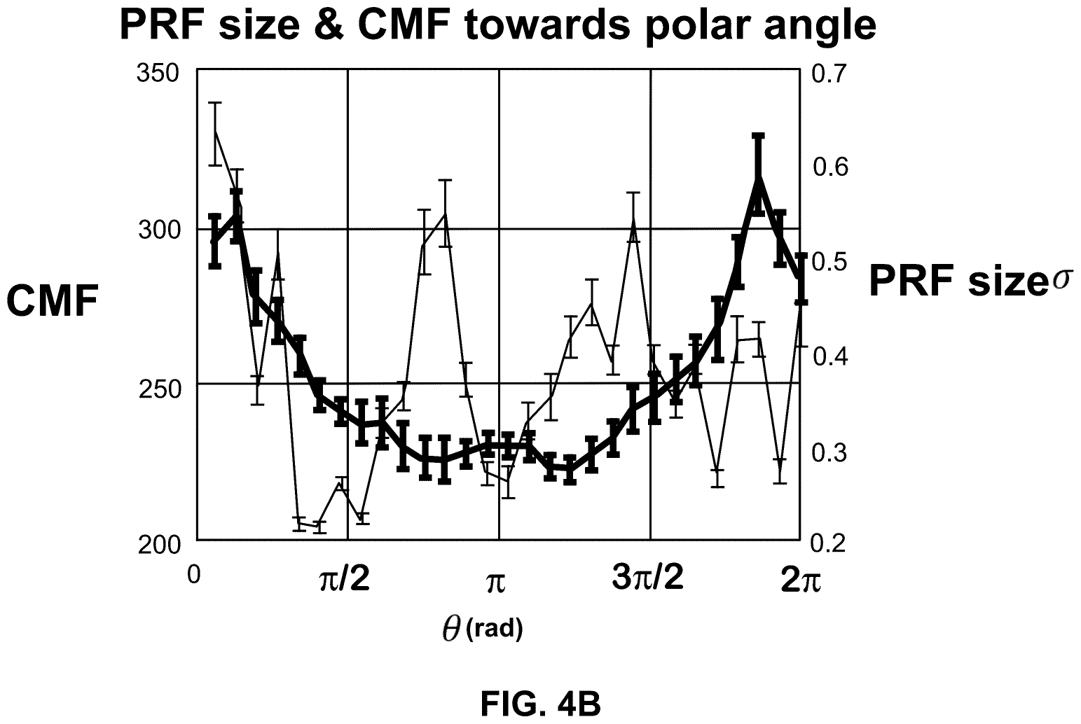

With reference now to FIGS. 4A and 4B, via operation of exemplary embodiments it can be seen there exists a negative correlation between pRF size σ and CMF. As our point moves outwards from the fovea, its eccentricity would increase, which accompanies the increase of pRF size σ and the decrease of CMF. This phenomenon accords with intuition: visual acuity in visual field reaches its highest at the fovea, and goes down for areas that are far away from the fovea.

In contrast, pRF size σ and CMF spread evenly across different polar angles. This phenomenon also accords with intuition: visual acuity in visual field reaches its highest at the fovea, and goes down for areas that are far away from the fovea.

Prior approaches supposed a negative correlation between pRF size and CMF across some variable such as eccentricity, polar angle, or distance to fovea. However, via use of an exemplary method, the results of pRF decoding can be projected onto the visual field, where CMF could also be calculated. To this extent, exemplary approaches are able to analyze the correlation between pRF response and CMF in the 2D planar visual domain.

### Individual pRF and CMF Correlation

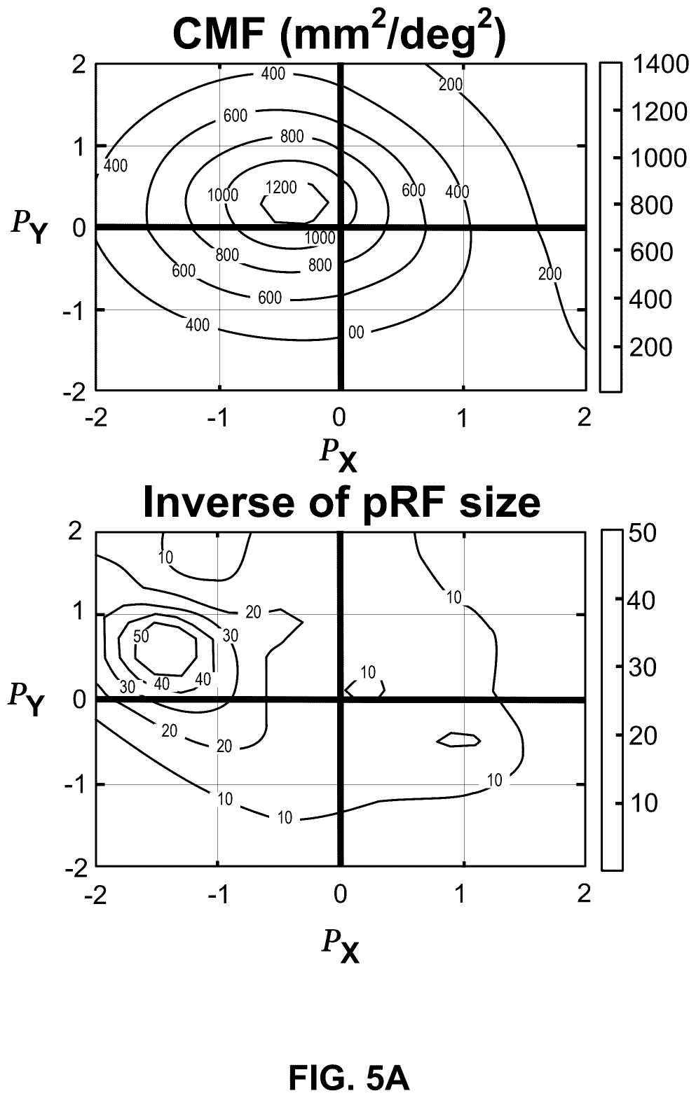

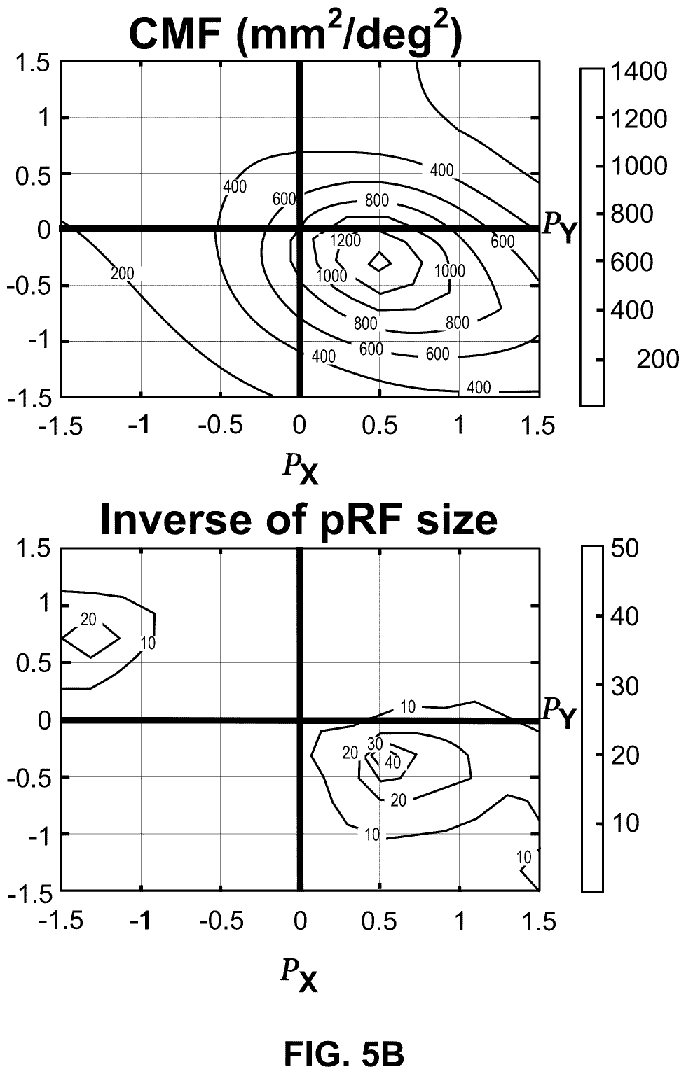

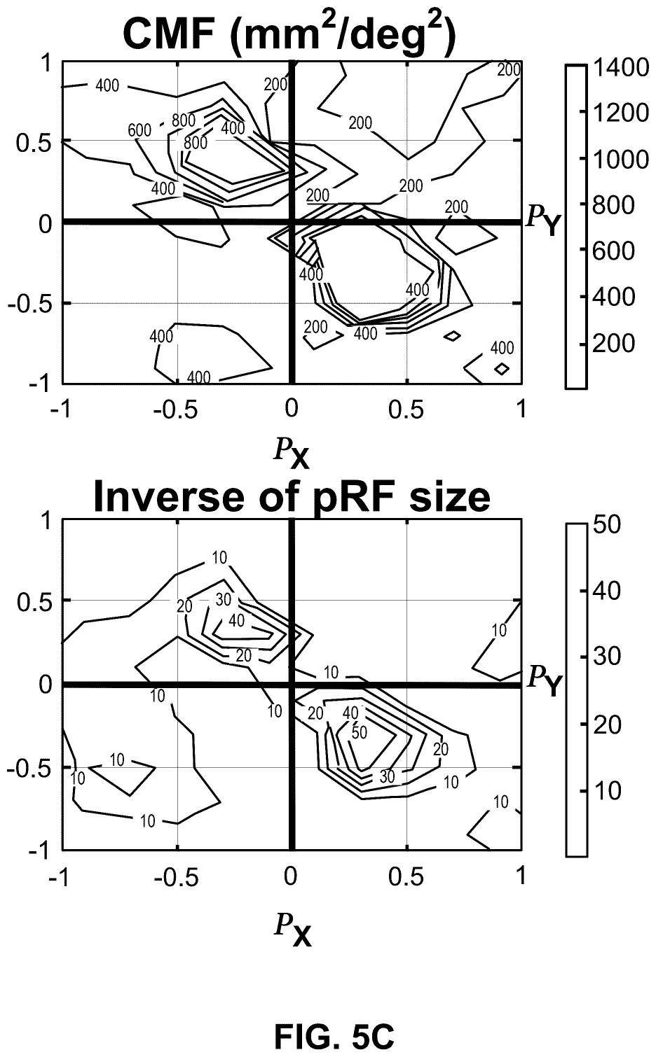

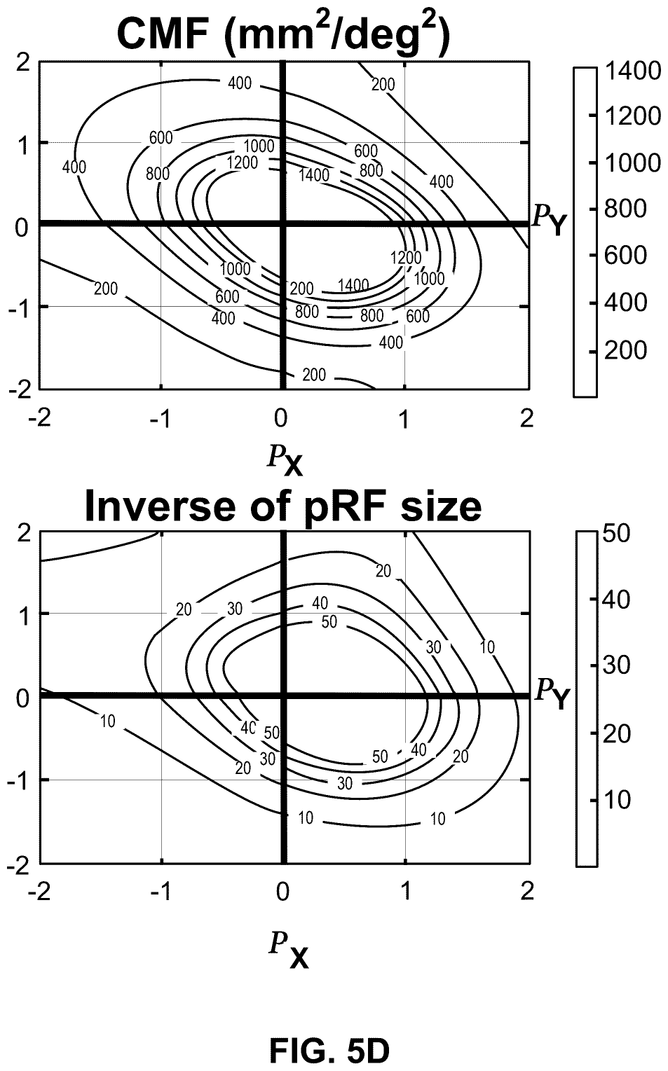

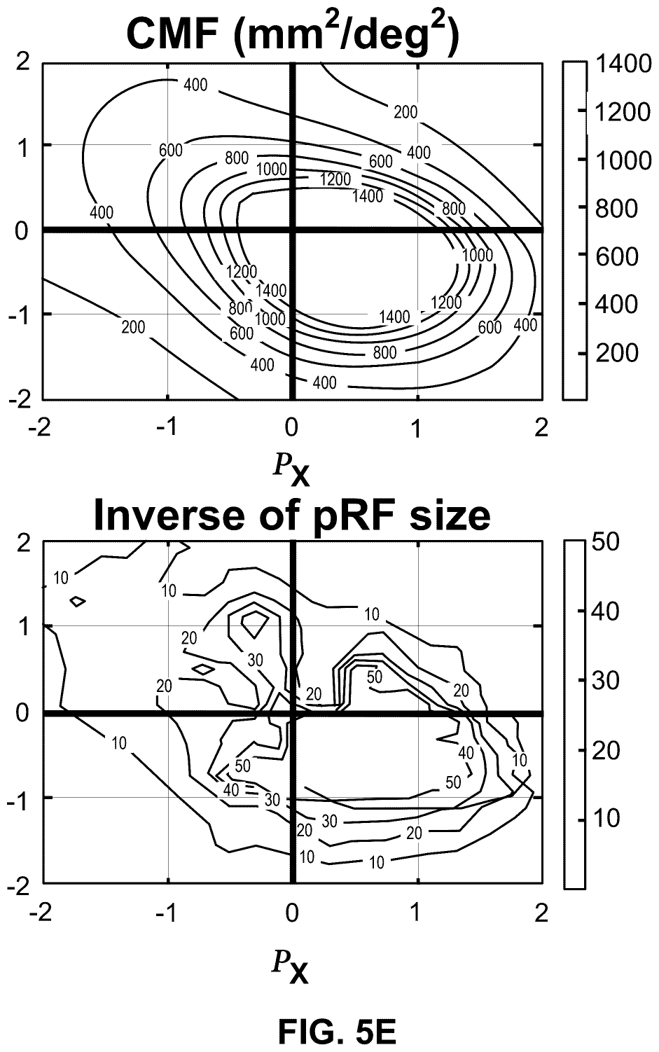

In addition to the foregoing, exemplary systems also analyzed population receptive field along with CMF in the subjects' visual field. The results are shown in FIGS. 5A through 5E, and it can be seen these CMF results give good alignment with the inverse of pRF size 1/σ around the 2D planar visual field.

The common understanding was that smaller pRF size or larger CMF represents better acuity around a particular point, and acuity decreases when eccentricity grew larger, which means points further from fovea would normally have higher pRF size and smaller CMF. In the examples disclosed herein, the trend of pRF in the planar coordinate was illustrated, which not only decreases along eccentricity growth, but also changes along polar angle.

It will be appreciated that for most subjects, the inverse of pRF size 1/σ correlates with the CMF behavior class. For example, a dual-centralized pattern in both planar CMF and 1/σ can be seen for subject 170. This phenomenon demonstrates that the exemplary clustering approach and algorithms could be tested in both CMF and pRF measurements; as can be seen, the individual behavior difference is illustrated in both 2 measurements.

### Further Discussion

Compared to current methods, exemplary “pipeline” methods are able to measure planar CMF in 2D visual fields. This enables a new approach to visualizei human vision and concentration through the measurement of CMF.

Exemplary embodiments disclose a novel pipeline to measure planar CMF on the whole visual field from structure MRI and fMRI scan. In various exemplary embodiments, example data show regarding measurement and visualization shows that individuals have different concentration area(s). To our knowledge, the topological smoother disclosed here is the first method that guarantees topological conditions in retinotopic mapping. Based on this foundation, planar CMF measurement were enabled, which contributes a new gauging approach for future research on human vision and retinotopy.

Recently, many researchers have shown asymmetries of cortical magnification in the human visual cortex, and their different performance across individuals with different twin status or gender. However, ordinary analysis showed the asymmetries but did not provide sufficient clarity. The exemplary findings disclosed herein show that exemplary systems may move one or more steps further than measuring average CMF, but illustrating the planar CMF distribution to see exactly the asymmetries and their change across individuals.

The exemplary results disclosed herein illustrate that the individual difference of CMF is much greater than anticipated. CMF of people varies in strength, concentration area, and spreading.

In various exemplary embodiments, pipeline 100 may be implemented utilizing an x64-based workstation computer using the Windows 10 operating system, having an Intel Xeon E3-1241 CPU operative at 3.5 GHz, 16 gigabytes of installed random access memory, and an Nvidia brand Quadron K620 graphics card. In various exemplary embodiments, Matlab software version R2022a may be utilized to implement the desired processing and computation steps. One or more of the following software packages may also be utilized: 1. Gifti toolbox (github.com/gllmflndn/gifti.git), 2. FreeSurfer Matlab toolbox (surfer.nmr.mgh.harvard.edu/), 3. AnalyzePRF Matlab toolbox (kendrickkay.net/analyzePRF/), 4. Fast Marching toolbox (www.mathworks.com/matlabcentral/fileexchange/6110-toolbox-fast-marching), and 5.

Geometry Processing package (www.mathworks.com/matlabcentral/fileexchange/46540-geometry-processing-package). However, it will be appreciated that pipeline 100 may be utilized in various other software environments or tools (such as Julia, GNU Octave, SciLab, or Sage) and/or utilizing other computational resources (such as a Linux, Unix, Mac, or similar workstation, personal computer, laptop, tablet computer, mainframe, or the like). Additionally, it will be appreciated that pipeline 100 may be constructed utilizing distributed software and/or computational resources, cloud computing, software as a service (SaaS) tools, and/or the like.

While the principles of this disclosure have been shown in various embodiments, many modifications of structure, arrangements, proportions, the elements, materials and components, used in practice, which are particularly adapted for a specific environment and operating requirements may be used without departing from the principles and scope of this disclosure. These and other changes or modifications are intended to be included within the scope of the present disclosure and may be expressed in the following claims.

The present disclosure has been described with reference to various embodiments. However, one of ordinary skill in the art appreciates that various modifications and changes can be made without departing from the scope of the present disclosure. Accordingly, the specification is to be regarded in an illustrative rather than a restrictive sense, and all such modifications are intended to be included within the scope of the present disclosure. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, required, or essential feature or element of any or all the claims.

As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. When language similar to “at least one of A, B, or C” or “at least one of A, B, and C” is used in the claims or specification, the phrase is intended to mean any of the following: (1) at least one of A; (2) at least one of B; (3) at least one of C; (4) at least one of A and at least one of B; (5) at least one of B and at least one of C; (6) at least one of A and at least one of C; or (7) at least one of A, at least one of B, and at least one of C.

What is claimed is:

1. A computer-based method for planar cortical magnification measurement, comprising:
obtaining a first input comprising a visual stimuli and a coordinate system of a visual field;
obtaining a second input comprising a structural magnetic resonance imaging (MRI) image and functional MRI (fMRI) scans;
storing the first input and the second input in an electronic database;
pre-processing, by a processor, the fMRI scans;
calculating, by the processor, a reconstructed cortical surface with projected fMRI activation and surface extraction;
calculating, by the processor, retinotopic maps comprising receptive center and population receptive field (pRF) size of each vertex, together with projection of the cortex onto a 2D planar disk with optimal transportation that preserves local area; and
applying, by the processor, topological smoothing to the calculated retinotopic maps to obtain smoothed retinotopic maps.

2. The method of claim 1, further comprising applying, by the processor, a 1-ring patch method to the smoothed retinotopic maps.

3. The method of claim 2, further comprising providing, to a medical provider and over an electronic communications network, the planar cortical magnification measurement.

4. The method of claim 1, further comprising utilizing, by the medical provider, the planar cortical magnification measurement to facilitate a medical diagnosis.

5. The method of claim 1, wherein the calculating the reconstructed cortical surface comprises acquiring a 3D cortical surface mesh.

6. The method of claim 1, wherein the calculating retinotopic maps comprises utilizing an experiment stimulus and fMRI signal into a pRF analysis module operative on the processor to obtain initial pRF results.

7. The method of claim 1, wherein the applying topological smoothing utilizes the initial pRF results as inputs.

8. The method of claim 2, wherein the reconstructed cortical surface comprises a left hemisphere and a right hemisphere.

9. The method of claim 8, wherein the calculating retinotopic maps and the applying topological smoothing are performed separately for the left hemisphere and the right hemisphere.

10. The method of claim 1, wherein the method is implemented as software code operative on the processor.
